(19) United States
(12) Patent Application Publication
(10) Pub. No.: US 2024/0115470 A1
(43) Pub. Date: Apr. 11, 2024

(54) METHYL-FLUORO-HYDROXYBENZOATES IN CONSUMER PRODUCTS

(71) Applicant: GIVAUDAN SA, VERNIER (CH)

(72) Inventors: Agnes BOMBRUN, Zürich (CH); Julie CHARPENTIER, Basel (CH)

(21) Appl. No.: 18/252,750

(22) PCT Filed: Dec. 8, 2021
(86) PCT No.: PCT/EP2021/084819

§ 371 Date: May 12, 2023

(30) Foreign Application Priority Data
Dec. 10, 2020 (GB) 2019524.4

(51) Int. Cl. A61K 8/37; A61Q 11/00; C11B 9/00

(52) U.S. Cl. CPC A61K 8/37; A61Q 11/00; C11B 9/0061

(57) ABSTRACT

Disclosed is the use of certain methyl-fluoro-hydroxybenzoates as flavor and fragrance constituents and for imparting a wintergreen odor note to a consumer product. Furthermore the use of certain methyl-fluoro-hydroxybenzoates as complete or partial replacement for methyl salicylate is disclosed.

### TECHNICAL FIELD

The present invention is concerned generally to consumer products comprising certain methyl-fluoro-hydroxybenzoates. The present invention further relates to the use of certain methyl-fluoro-hydroxybenzoates for imparting a wintergreen odor note to a consumer product. The present invention further relates to the use of certain methyl-fluoro-hydroxybenzoates as complete or partial replacement for methyl salicylate.

### BACKGROUND

Methyl salicylate is a commonly used flavour and fragrance compound which provides the characteristic flavour of wintergreen to foods, beverages, candies, chewing gums and oral care products. The sensory properties of methyl salicylate are described as wintergreen, sweet, warm, slightly phenolic.

While products flavored with methyl salicylate have gained widespread consumer acceptance, the use of such a compound poses a potential risk (CMR 2) of adverse publicity or governmental regulation which could limit the level at which it may be used, or prohibit it entirely.

The availability of a replacement compound for methyl salicylate would be highly desirable.

### SUMMARY OF THE INVENTION

In accordance with the first aspect of the present invention there is provided the use as flavor or fragrance a methyl-fluoro-hydroxybenzoate selected from methyl 4-fluoro-2-hydroxybenzoate and methyl 2-fluoro-6-hydroxybenzoate.

In accordance with the second aspect of the present invention there is provided a consumer product comprising methyl-fluoro-hydroxybenzoate selected from methyl 4-fluoro-2-hydroxybenzoate and methyl 2-fluoro-6-hydroxybenzoate.

In accordance with a third aspect of the present invention there is provided the use of methyl-fluoro-hydroxybenzoate as a wintergreen flavor imparting ingredient, characterized in that the methyl-fluoro-hydroxybenzoate is selected from methyl 4-fluoro-2-hydroxybenzoate and methyl 2-fluoro-6-hydroxybenzoate.

### DETAILED DESCRIPTION

Methyl-fluoro-hydroxybenzoates are well known and are widely used as starting materials in the synthesis of pharmaceuticals and agrochemicals.

It has now surprisingly been found that both, methyl 4-fluoro-2-hydroxybenzoate and methyl 2-fluoro-6-hydroxybenzoate possessing an odor profile which is very similar to methyl salicylate. Beside the wintergreen odor characteristic, the sweetness aspect of methyl salicylate plays an important role to the overall sensory profile. Whereas both, methyl 4-fluoro-2-hydroxybenzoate and methyl 2-fluoro-6-hydroxybenzoate possessing these sweet aspect, theses aspects are essentially missing for methyl 3/5-fluoro-2-hydroxybenzoate.

Furthermore both methyl-fluoro-hydroxybenzoates (methyl 4-fluoro-2-hydroxybenzoate and methyl 2-fluoro-6-hydroxybenzoate) possessing surprisingly low odor threshold concentrations compared to methyl salicylate and compounds with other methyl-fluoro-hydroxybenzoates (such as methyl 3/5-fluoro-2-hydroxybenzoate). Due to the surprisingly low odor threshold of methyl 4-fluoro-2-hydroxybenzoate and methyl 2-fluoro-6-hydroxybenzoate compared to methyl salicylate, less can be used to achieve a similar odor profile.

Accordingly, there is provided in a first aspect of the present invention the use as flavour or fragrance of a methyl-fluoro-hydroxybenzoate selected from methyl 4-fluoro-2-hydroxybenzoate and methyl 2-fluoro-6-hydroxybenzoate.

In one particular embodiment methyl 4-fluoro-2-hydroxybenzoate and/or methyl 2-fluoro-6-hydroxybenzoate is used to impart wintergreen olfactory properties to a fragrance flavor composition.

Due to the similar odor profile of methyl 4-fluoro-2-hydroxybenzoate and methyl 2-fluoro-6-hydroxybenzoate compared to methyl salicylate, said compounds can be used to fully or partly replace methyl salicylate in flavor and/or fragrance compositions without forgoing the sought after wintergreen olfactory profile.

Thus there is provided in a further aspect a flavor or fragrance composition comprising methyl 4-fluoro-2-hydroxybenzoate and/or methyl 2-fluoro-6-hydroxybenzoate, characterized in that the composition is essentially free of methyl salicylate.

By a flavor or fragrance composition “essentially free of methyl salicylate”, a composition is meant preferably comprising 0.005 wt % or less (e.g. 0.004, 0.003, 0.002, 0.001. 0.0005, 0.0001, 0.00005, 0.00001 or less) based on said composition.

The fragrance/flavour composition of the first aspect of the invention may be admixed by any means to a consumer product.

As specific examples of consumer products one may mention products such as fine perfumery, e.g. perfume and eau de toilette; fabric care, household products and personal care products such as oral care products, cosmetics, laundry care detergents, rinse conditioner, personal cleansing composition, detergent for dishwasher, surface cleaner; laundry products, e.g. softener, bleach, detergent; body-care products, e.g. shampoo, shower gel; air care products, e.g. air-freshener, and the like. This list of products is given by way of illustration, and is not to be regarded as being in any way limiting.

In one particular embodiment the consumer product is consumable. According to the present disclosure, the term “consumables” refers to products for consumption by a subject, typically via the oral cavity (although consumption may occur via non-oral means such as inhalation), for at least one of the purposes of enjoyment, nourishment, or health and wellness benefits. Consumables may be present in any form including, but not limited to, liquids, solids, semi-solids, tablets, capsules, lozenges, strips, powders, gels, gums, pastes, slurries, syrups, aerosols and sprays. The term also refers to, for example, dietary and nutritional supplements. Consumables include compositions that are placed within the oral cavity for a period of time before being discarded but not swallowed. It may be placed in the mouth before being consumed, or it may be held in the mouth for a period of time before being discarded.

Broadly, consumables include, but are not limited to, foodstuffs of all kinds, confectionery products, baked products, sweet products, savory products, fermented products, dairy products, beverages, oral care products, nutraceuticals and pharmaceuticals.

The term “oral care products” as used herein refers to non-food compositions that are designed to be taken into the mouth to deliver a variety of benefits. Such compositions include dentifrices, mouthwashes, mouth sprays and gargle compositions, breath strips (edible films placed in the oral cavity to administer thereto an active agent such as a flavourant or breath-freshening agent), and chewing gums. The term “dentifrice”, as used herein, means toothpaste, oral care gels or liquids, unless otherwise specified. The dentifrice composition may be a single-phase composition or it may be a combination of two or more separate dentifrice compositions. The dentifrice composition may be in any desired form, such as deep-striped, surface-striped, multilayered, having the gel surrounding the paste, or any combination thereof.

The oral care products typically comprises further ingredients which are common in the art, such as, odorants and flavors (other than methyl 4-fluoro-2-hydroxybenzoate and methyl 2-fluoro-6-hydroxybenzoate), sweeteners, cooling agents, antimicrobial agents, anti-inflammatory agents, anti-caries agents, abrasives, plaque puffers, humectants, vitamins, plant extracts, desensitising agents, anti-calculus agents, preservatives, coloring agents, pH-adjusting agents, surfactants, and the like.

Methyl 4-fluoro-2-hydroxybenzoate and/or methyl 2-fluoro-6-hydroxybenzoate may be employed in widely varying amounts, depending on the specific product and on the nature and quantity of the other odorant ingredients present. The proportion is typically from 0.00001 to 1 weight percent of the product. In one embodiment, methyl 4-fluoro-2-hydroxybenzoate and/or methyl 2-fluoro-6-hydroxybenzoate may be employed in a an oral care products in an amount from 0.1 to 0.005 wt % (e.g. toothpaste about 0.0001-0.05 wt %, or mouthwash 0.00005 to 0.01 wt %). However, these values are given only by way of example, since the experienced perfumer/flavorist may also achieve effects or may create novel odor profiles with lower or higher concentrations.

Methyl 4-fluoro-2-hydroxybenzoate and/or methyl 2-fluoro-6-hydroxybenzoate may be used alone or blended with, e.g., flavor oils, such as spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents include artificial, natural and synthetic fruit flavors such as vanilla, and citrus oils including lemon, orange, lime, grapefruit, yuzu, sudachi, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth.

In another embodiment methyl 4-fluoro-2-hydroxybenzoate and/or methyl 2-fluoro-6-hydroxybenzoate may be blended with cooling agents, such as, p-menthanecarboxamides, N-2,3-trimethyl-2-isopropyl-butanamide (WS-23), menthyl lactate (Frescolat® ML), menthone glycerol acetal (Frescolat® MGA), 3-(1-menthoxy)-propane-1,2-diol (TK-10), p-menthane-3,8-diol (known as Coolact 38D), isopulegol (known as Coolact P), monomenthyl succinate (Physcool®), monomenthyl glutarate, o-menthylglycerol, menthyl N,N-dimethylsuccinamate, 2-(sec-butyl)cyclohexan-1-one (Freskomenthe), N-(pyrazol-3-yl)-N-(thiophen-2-ylmethyl)-2-(p-tolyloxy)acetamide, 2-(4-ethylphenoxy)-N-(pyrazol-3-yl)-N-(thiophen-2-ylmethyl)acetamide, 3-(benzo[d][1,3]dioxol-5-yl)-N,N-diphenylacrylamide, 4-(2-(4-allyl-2,6-dimethoxyphenoxy)-1-ethoxypropyl)-2-methoxyphenol, 4-(2-(4-allyl-2,6-dimethoxyphenoxy)-1-((2-isopropyl-5-methylcyclohexyl)oxy)propyl)-2-methoxyphenol (including 4-(2-(4-allyl-2,6-dimethoxyphenoxy)-1-(((1S,2R,5S)-2-isopropyl-5-methylcyclohexyl)oxy)propyl)-2-methoxyphenol) and 4-(2-(4-allyl-2,6-dimethoxyphenoxy)-1-(((1R,2S,5R)-2-isopropyl-5-methylcyclohexyl)oxy)propyl)-2-methoxyphenol), N-(2-Hydroxy-2-phenylethyl)-2-isopropyl-5,5-dimethylcyclohexane-1-carboxamide, N-(4-(Cyanomethyl)phenyl)-2-isopropyl-5,5-dimethylcyclohexanecarboxamide, N-(3-Hydroxy-4-methoxyphenyl)-2-isopropyl-5,5-dimethylcyclohexanecarboxamide, 2-methyl-1-(2-(5-(p-tolyl)-1H-imidazol-2-yl)piperidin-1-yl)butan-1-one, 2-(methylthio)-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, 2,2-dimethyl-1-(2-(5-(p-tolyl)-1H-imidazol-2-yl)piperidin-1-yl)but-3-en-1-one, 1-isopropyl-3,3,5,7-tetramethyloctahydrobenzo[c]isoxazole, 5-(4-fluorophenyl)-1,3,3,5,7-pentamethyloctahydrobenzo[c]isoxazole, and 4-methyl-3-(1,3,3,5,7-pentamethyloctahydrobenzo[c]isoxazol-5-yl)benzonitrile.

Hereinafter, the present invention will be described in further detail with reference to examples. It is to be understood, however, that these examples are for illustrative purposes only and are not to be construed to limit the scope of the present invention.

### Example 1: methyl-fluoro-hydroxybenzoates

1.1: methyl 4-fluoro-2-hydroxybenzoate was obtained from Sigma Aldrich.

The sample was purified by bulb-to-bulb distillation in a Büchi Kugelrohr B-585 at 150° C. and 0.050 mbar.

¹H-NMR (400 MHz, CDCl₃): 10.99 (d, J=1.5 Hz, 1H), 7.83 (dd, J=6.6, 8.8 Hz, 1H), 6.66 (dd, J=2.6, 10.4 Hz, 1H), 6.59 (ddt, J=0.7, 2.4, 8.6 Hz, 1H), 3.94 (s, 3H).

¹³C-NMR (101 MHz, CDCl₃): 169.93 (s, 1 C), 167.21 (d, J=254.59 Hz, 1 C), 163.69 (d, J=14.10 Hz, 1 C), 132.05 (d, J=11.61 Hz, 1 C), 109.09 (d, J=2.49 Hz, 1 C), 107.30 (d, J=23.22 Hz, 1 C), 104.34 (d, J=24.05 Hz, 1 C), 52.32 (s, 1 C).

MS (EI, 70 eV): 170 (43, [M]+.), 139 (41), 138 (100), 111 (12), 110 (60), 83 (22), 82 (16), 81 (9), 57 (15), 29 (8).

1.2: methyl 2-fluoro-6-hydroxybenzoate was obtained from Apollo Scientific.

The sample was purified by bulb-to-bulb distillation in a Büchi Kugelrohr B-585 at 120° C. and 0.07 Torr (0.093 mbar).

¹H-NMR (400 MHz, CDCl₃): 11.27 (s, 1H), 7.38 (td, J=8.38, 5.99 Hz, 1H), 6.80 (dt, J=8.50, 1.01 Hz, 1H), 6.62 (ddd, J=11.00, 8.19, 1.10 Hz, 1H), 4.00 (s, 3H).

¹³C-NMR (101 MHz, CDCl₃): 169.89 (d, J=4.15 Hz, 1 C), 163.65 (s, 1 C), 163.02 (s, 1 C), 162.02 (d, J=194.88 Hz, 1 C), 135.21-135.54 (m, 1 C), 113.33 (d, J=4.15 Hz, 1 C), 106.93 (d, J=23.22 Hz, 1 C), 101.74-103.09 (m, 1 C), 52.69 (s, 1 C).

MS (EI, 70 eV): 170 (40, [M]+.), 139 (41), 138 (100), 111 (10), 110 (86), 83 (20), 82 (14), 81 (8), 63 (8), 57 (18).

1.3: methyl 3-fluoro-2-hydroxybenzoate was obtained from Apollo Scientific.

The sample was purified by bulb-to-bulb distillation in a Büchi Kugelrohr B-585 at 150° C. and 0.080 mbar.

¹H-NMR (400 MHz, CDCl₃): 10.81 (s, 1H), 0.00 (td, J=1.5, 8.1 Hz, 1H), 7.28 (ddt, J=0.5, 2.0, 9.0 Hz, 1H), 6.83 (dt, J=4.5, 8.1 Hz, 1H), 3.98 (s, 3H).

¹³C-NMR (101 MHz, CDCl₃): 170.10 (s, 1 C), 151.50 (d, J=236.34 Hz, 1 C), 150.21 (d, J=3.31 Hz, 1 C), 124.86 (d, J=3.32 Hz, 1 C), 121.43 (d, J=17.42 Hz, 1 C), 118.39 (d, J=6.63 Hz, 1 C), 114.46 (s, 1 C), 52.60 (s, 1 C).

MS (EI, 70 eV): 170 (30, [M]+.), 139 (27), 138 (100), 111 (7), 110 (28), 83 (15), 82 (14), 81 (7), 57 (10), 29 (7).

1.4: methyl 5-fluoro-2-hydroxybenzoate was obtained from Apollo Scientific.

The sample was purified by bulb-to-bulb distillation in a Büchi Kugelrohr B-585 at 150° C. and 0.083 mbar.

¹H-NMR (400 MHz, CDCl₃): 10.52 (s, 1H), 7.51 (dd, J=3.2, 9.0 Hz, 1H), 7.20 (ddd, J=3.2, 7.9, 8.9 Hz, 1H), 6.95 (dd, J=4.5, 9.2 Hz, 1H), 3.97 (s, 3H).

¹³C-NMR (101 MHz, CDCl₃): 169.73 (d, J=2.49 Hz, 1 C), 157.84 (s, 1 C), 157.06 (d, J=152.59 Hz, 1 C), 123.26 (d, J=23.22 Hz, 1 C), 118.83 (d, J=7.46 Hz, 1 C), 115.04 (d, J=24.05 Hz, 1 C), 112.28 (d, J=7.46 Hz, 1 C), 52.55 (s, 1 C).

MS (EI, 70 eV): 170 (39, [M]+.), 139 (32), 138 (100), 111 (13), 110 (60), 83 (20), 82 (20), 81 (10), 57 (15), 29 (9).

### Example 2: Assessment of the Odor Profile

A 10 wt % solution of the purified samples of Example 1 were prepared (compound @10 wt % in polyethylene glycol). The odor profile was assessed freshly dipped on smelling strips.

Panellists have been asked to provide an odor description as compared to methyl salicylate. The results are shown in the table below.

| compound | Odor description |
|---|---|
| methyl 4-fluoro-2- | Wintergreen, typical methyl salicylate |
| hydroxybenzoate | character, creamy, sweet. |
| methyl 2-fluoro-6- | Sweetness, elements of wintergreen, |
| hydroxybenzoate | heliotropine facet. |
| methyl 3-fluoro-2- | Medicinal note, technical |
| hydroxybenzoate | |
| methyl 5-fluoro-2- | less sweetness, methyl salicylate character |
| hydroxybenzoate | more medicinal and phenolic. Lacks intensity. |

### Example 3: GC-Odor Threshold Concentration

Definition: The odour threshold concentration is defined as the lowest concentration of the vapour of an odorant material in the air which can be detected by smell and can be measured by standard methods known in the art.

According to standard procedures known to the person skilled in the art, threshold concentrations for volatile perfumery compounds are determined on a gas chromatograph equipped with a sniff port by a panel of trained evaluators. The lowest concentration smelled by each panelist is recorded as the individual threshold concentration expressed in ng (absolute amount of compound delivered at the sniff port).

Under identical conditions the odor threshold concentration for

### Comparison Examples

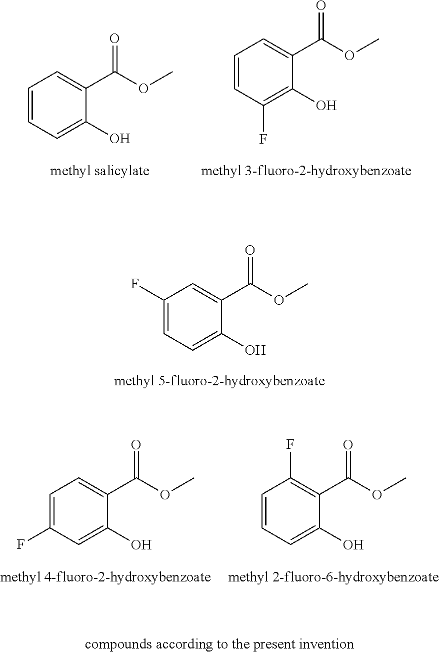

was measured. The results are shown below.

| | odor threshold |
|---|---|
| Compound | concentration (relative) |
| methyl 4-fluoro-2-hydroxybenzoate | 4 |
| methyl 2-fluoro-6-hydroxybenzoate | 1 |
| methyl 3-fluoro-2-hydroxybenzoate | 42 |
| methyl 5-fluoro-2-hydroxybenzoate | 36 |
| methyl 2-hydroxybenzoate (Methyl Salicylate) | 22 |

As can be seen from the results above, for example, the odor threshold concentration of methyl 4-fluoro-2-hydroxybenzoate is more than 5 times lower than the odor threshold concentration of methyl salicylate and more than 10 times lower than the odor threshold of methyl 3-fluoro-2-hydroxybenzoate.

### Example 4: Flavor Formulation Suitable for Toothpaste Product

| Compound/Ingredient | parts by weight 1/100 |
|---|---|
| ANETHOLE | 11.00 |
| CLOVE OILTERPENELESS | 1.46 |
| EUCALYPTUS GLOBULUS OIL | 3.00 |
| EUGENOL | 7.00 |
| LAEVO-MENTHOL | 51.30 |
| METHYL SALICYLATE | 20.00 |
| PEPPERMINT OIL | 6.00 |
| ROSEMARY OIL | 0.04 |
| SAFRALEINE ® (2,3,3-Trimethylindan-1-one) | 0.02 |
| TEA TREE OIL | 0.10 |
| THYMOL) | 0.04 |
| TOSCANOL ® (1-Cyclopropylmethyl- | 0.04 |
| 4-methoxybenzene) | |
| Total | 100 |

The formulation above is a typical wintergreen type flavor suitable for the use in toothpaste products, at a dosage of, e.g. 1.2 wt %.

The replacement of 20 parts methyl salicylate by 0.2 parts methyl 4-fluoro-2-hydroxybenzoate and 19.8 parts PG (Propylene Glycol) results in a profile with very similar characteristics.

### Example 5: Flavor Formulation Suitable for Mouthwash Product

| Compound/Ingredient | parts by weight 1/100 |
|---|---|
| ANETHOLE | 1.8 |
| LAEVO-CARVONE | 1.0 |
| EUCALYPTOL | 30.7 |
| LEAVO-MENTHOL | 14.0 |
| METHYL SALICYLATE | 20.0 |
| PEPPERMINT ARVENSIS | 3.5 |
| PEPPERMINT PIPERITA | 6.0 |
| SPEARMINT OIL | 2.0 |
| THYMOL | 21.0 |
| Total | 100 |

The formulation above is a typical wintergreen type flavor suitable for the use in mouthwash products, at a dosage of, e.g. 0.3 wt %.

The replacement of 20 parts methyl salicylate by 0.2 parts methyl 4-fluoro-2-hydroxybenzoate and 19.8 parts PG (propylene glycol) results in a profile with very similar characteristics.

### Example 6: Fragrance Formulation

| | parts by |
|---|---|
| Compound/Ingredient | weight 1/1000 |
| ANAPEAR¹⁾ @10% DPG | 5 |
| ANATOLYL (2-PHENYLETHYL 2- | 5 |
| METHYLBUTANOATE) | |
| BENZYL SALICYLATE | 450 |
| DAMASCENONE @ 10% DPG | 1 |
| DIHYDRO FARNESAL (3,7,11-TRIMETHYL-6,10- | 20 |
| DODECADIENAL) | |
| BETA DIHYDRO IONONE | 1 |
| GARDENOL (1-PHENYLETHYL ACETATE) | 4 |
| HEDIONE HC ²⁾ | 10 |
| CIS-3-HEXENOL @ 10% DPG | 2 |
| CIS-3-HEXENYL ACETATE @ 10% DPG | 2 |
| CIS-3-HEXENYL BENZOATE | 40 |
| CIS-3-HEXENYL BUTYRATE @ 10% DPG | 5 |
| CIS-3-HEXENYL HEXENOATE | 1 |
| CIS-3-HEXENYL SALICYLATE | 100 |
| INDOLE (2,3-BENZOPYROLE) | 7 |
| BETA IONONE | 2 |
| CIS JASMONE (3-methyl-2-(pent-2-en-1-yl)cyclopent- | 1 |
| 2-enone) | |
| LEMON OIL | 3 |
| LINALOOL OXIDE | 3 |
| LINALOOL | 10 |
| METHYL ANTHRANILATE (METHYL 2- | 1 |
| AMINOBENZOATE) | |
| MYRALDYL ACETATE ³⁾ | 50 |
| NEROLIDOL⁴⁾ | 35 |
| OCIMENE (3,7-DIMETHYL-1,3,6-OCTATRIENE) | 4 |
| DIPROPYLENE GLYCOL (DPG) | 238 |
| Total | 1000 |

¹⁾Methyl (4E)-4,7-octadienoate
²⁾ cyclopentaneacetic acid, 3-oxo-2-pentyl, methylester
³⁾ 3/4-(4-METHYLPENTEN-3-YL)CYCLOHEX-3-ENE-1-METHYLACETATE
⁴⁾6-TRANS-3,7,11-TRIMETHYL-1,6,10-DODECATRIEN-3-OL

The accord above represents a light floral cologne accord, suitable for fine perfumery at a dosage of e.g. 5 weight %.

The replacement of 4 parts DPG by methyl salicylate helps to reinforce the fresh, transparent, natural top note. Without methyl salicylate, the accord lacks initial freshness and neutrality on the top note.

The replacement of 4 parts DPG by methyl 4-fluoro-2-hydroxybenzoate results in an accord with a fresh and green natural effect on the top note. Thus an accord was obtained which compensates the effects normally achieved by the presence of methyl salicylate, thus a useful compensation of methyl salicylate is obtained.

1. A consumer product comprising a methyl-fluoro-hydroxybenzoate selected from methyl 4-fluoro-2-hydroxybenzoate and methyl 2-fluoro-6-hydroxybenzoate.

2. The consumer product according to claim 1, wherein the product is essentially free of methyl salicylate.

3. The consumer product according to claim 1 wherein the consumer product is consumable.

4. A flavor or fragrance composition comprising a methyl-fluoro-hydroxybenzoate selected from methyl 4-fluoro-2-hydroxybenzoate and methyl 2-fluoro-6-hydroxybenzoate

5. A flavour or fragrance composition which imparts a wintergreen flavor comprising a methyl-fluoro-hydroxybenzoate selected from methyl 4-fluoro-2-hydroxybenzoate and methyl 2-fluoro-6-hydroxybenzoate.

6. The consumer product according to claim 2 wherein the consumer product is consumable.
